(19) United States
(12) Patent Application Publication
(10) Pub. No.: US 2006/0134195 A1
(43) Pub. Date: Jun. 22, 2006

(54) Mannose-based fast dissolving tablets

(76) Inventors: Yourong Fu, West Lafayette, IN (US); Seong Hoon Jeong, West Lafayette, IN (US); Jeanny Kim, West Lafayette, IN (US); Jacqueline A. Callihan, West Lafayette, IN (US); Kinam Park, West Lafayette, IN (US); Chaul Min Pai, Daejeon (KR); Sang Yeob Park, Daejeon (KR); Gun Seomoon, Daejeon (KR)

Correspondence Address: JAMES H. MEADOWS AND MEDICUS ASSOCIATES, 2804 KENTUCKY, JOPLIN, MO, 64804, US

(21) Appl. No.: 10/536,197

(22) PCT Filed: Nov. 25, 2003
(86) PCT No.: PCT/US03/38145

§ 371 Date: May 24, 2005

Related U.S. Application Data
(60) Provisional application No. 60/429,026, filed on Nov. 25, 2002.

(51) Int. Cl. A61K 9/20

(52) U.S. Cl. 424/464

(57) ABSTRACT

Fast-dissolving pharmaceutical tablets comprising mannose are described. The mannose component imparts both structure-forming and fast-dissolution properties to the tablets. Granulation of tablet components and humidification forms strong liquid bridges at the surface interfaces of mannose particles, which leads to strengthened tablets. The mannose particles, however, remain porous following compression so that contact with moisture, e.g., saliva in the mouth, leads rapidly to tablet disintegration and dissolution.

### REFERENCE TO RELATED APPLICATION

This application claims the benefit of U.S. Provisional Application 60/429,026, filed Nov. 25, 2002.

### BACKGROUND OF THE INVENTION

A. Fast Dissolving Tablets

Although a conventional tablet is the most popular drug dosage form, its administration still causes problems for some populations, such as the elderly and children, who have difficulties in swallowing tablets. During the last decade, fast-dissolving tablet technologies that make tablets disintegrate in the mouth without additional water uptake have drawn a great deal of attention. Fast-dissolving tablets are also called fast-disintegrating tablets or fast-melting tablets. The name “fast-dissolving” indicates that the tablets dissolve fast in the mouth. It also indicates that the tablets disintegrate into smaller granules, or melt in the mouth from a hard solid structure to gel-like structures allowing easy swallow by the patients. Currently, fast-dissolving tablets are prepared by several approaches as listed in Table 1.

TABLE 1
Technologies used in the preparation of fast-dissolving tablets.

| | | Advantages | Disadvantages |
|---|---|---|---|
| 1. | Freeze drying | Dissolve within | Highly fragile, |
| | | seconds | expensive |
| 2. | Molding | Low pressure for | Poor mechanical |
| | | making tablets | strength |
| 3. | Sublimation | No pressure for | Use of volatile |
| | | making tablets | materials |
| 4. | Direct compression | High mechanical | Slow |
| | | strength, low cost | disintegration |

Freeze drying (lyophilization) is a process in which water is sublimated from a drug solution or suspension containing structure forming excipients after freezing. Freeze drying results in amorphous and porous structure necessary for fast disintegration/dissolution (1). When placed on the tongue the unit dissolves almost instantly to release the incorporated drug. Because the freeze drying process avoids elevated temperatures, no adverse thermal effects are expected on the incorporated drugs. Freeze drying, however, is a relatively expensive manufacturing process and the final dosage forms are very fragile, lacking physical resistance in standard blister packs. Moreover, this approach does not allow accommodating high amounts of active drugs. Also, the formulation has poor stability at higher temperature and humidity (2). ZYDIS (manufactured by R.P. Scherer, Basking Ridge, N.J.) was the first marketed tablet produced by the freeze-drying technology. ZYDIS formulations consist of a drug physically trapped in a matrix that is composed of a water-soluble mixture of saccharide (mannitol) and polymer (gelatin). Since the product is highly fragile, it has to be packaged in a special blister pack.

Molded tablets use water-soluble ingredients to achieve fast disintegration. The powder is first blended with hydroalcoholic solvent, and then the wet mass is molded into a tablet with pressure lower than that used in making conventional tablets. Removal of the solvent results in a porous structure (1). One of the disadvantages of this technology is the poor mechanical strength of molded tablets (3) [U.S. Pat. No. 5,082,667 to Van Scoik].

In sublimation technology, the high porosity necessary for fast disintegration is achieved by using volatile materials. Inert solid ingredients, such as urea, ammonium carbonate, ammonium bicarbonate, hexamethylene tetramine, and camphor, can volatilize readily. When these volatile materials are compressed into tablets, they can be removed via sublimation, which generates porous structures. In addition, several solvents (e.g., cyclohexane, benzene) can also be used as pore forming agents (1, 4) [U.S. Pat. No. 5,762,961 to Roser et al.].

Fast dissolving tablets produced by direct compression are based on the single or combined action of disintegrating agents, water-soluble excipients and effervescent agents. Disintegrants play a major role in the disintegration and dissolution processes. Choice of a suitable disintegrant at an optimal amount is critical for the fast dissolving property of resulting tablets. Addition of water-soluble excipients or effervescent agents to the formulation further improves the dissolution or disintegrating properties of the tablets (5). FLASHTAB technology (Laboratoires Prographarm, France) produces tablets by compression of granular excipients (6) [U.S. Pat. No. 5,464,632 to Cousin et al.]. Excipients used in this technology comprise two groups of components. One group is disintegrating agents, such as carboxymethylcellulose or insoluble reticulated polyvinylpyrrolidone (PVP). The other group is swelling agents, and examples are carboxymethylcellulose, starch, modified starch, carboxymethylated starch, microcrystalline cellulose, and possibly directly compressible sugars. The produced tablets are known to have satisfactory physical resistance and disintegrate in the mouth within 1 min.

ORASOLV technology (Cima Labs, Inc.) also produces tablets by low pressure compression (7) [U.S. Pat. No. 5,178,878 to Wehling et al.]. Excipients include a small amount of an effervescent couple that can be activated through saliva of a patient. Because of the soft and fragile nature of ORASOLV tablets, a special packaging system, known as PAKSOLVE (8) [U.S. Pat. No. 6,311,462 to Amborn et al.], was developed to protect the tablets from breaking during transport and storage. The DURASOLV technology was developed by the same company to provide stronger tablets for packaging in foil pouches or bottles (9) [U.S. Pat. No. 6,024,981 to Khankari et al.]. In addition to the active ingredients, the tablet excipients contain a “non-direct compressible filler” and a lubricant.

So-called WOWTAB technology (Yamanouchi Pharmaceutical Co.) proposes a combination of “low moldability” and “high moldability” saccharides to produce fast dissolving tablets using conventional granulation and tableting techniques (10) [U.S. Pat. No. 5,576,014 to Mizumoto et al.]. According to the patent describing the WOWTAB technology, saccharides having “low moldability” are those that produce tablets having a hardness between 0-2 kg when 150 mg of such a saccharide is compressed into a tablet under a pressure of 10-50 kg/cm² using a die of 8 mm in diameter. The saccharides having “high moldability” are those producing tablets with hardness of 2 kg or more when they are prepared under the identical condition. The patent indicates that no single raw material was found that makes tablets having both high strength and fast disintegration properties. For this reason, a saccharide having low moldability was granulated with a saccharide having high moldability as a binder. After the granules were compressed into tablets, the prepared tablets were further treated with moisture under a certain humidity condition to form liquid bridges between granules. Upon drying, the formed bridges reportedly increase the mechanical property of the dried tablet (11) [U.S. Pat. No. 6,589,554 to Mizumoto et al.].

As described above, several technologies are available for making fast disintegrating tablets, but each method has its limitations and disadvantages. In order to overcome those limitations and disadvantages, a new method of making fast melting tablets has been developed and is described hereinafter.

B. The Mechanisms of Granule Aggregation

The mechanical strength of a fast dissolving tablet depends on the binding strength of powders and/or granules constituting the tablet. The binding mechanisms of agglomeration have been classified into five groups (12). They are known as solid bridge formation by sintering, liquid bridge formation by surface tension and capillary forces, adhesion and cohesion forces, attraction force between solid particles consisting of molecular, electric and magnetic forces, and interlocking bonds. Any combination of the five bonding mechanisms can be used to make strong tablets.

When external mechanical forces are applied to a powder mass, the initial reduction in volume is through particle rearrangements. When the rearrangement becomes more difficult as load increases, further compression will result in some type of particle deformation. Two kinds of deformation are possible: elastic and plastic deformation. When subjected to compression, all solids experience more or less elastic deformation, which is reversible when the load is removed. When the yield point is reached, however, deformation will not be reversible on removal of the applied force. If shear strength is less than the tensile or breaking strength, plastic deformation is predominant. Plastic deformation usually creates good contacts between particles leading to good bonding. If the shear strength becomes greater, however, particles tend to become fractured to expose fresh surfaces for bonding (13).

C. Disintegration Mechanisms

Before a tablet dissolves, it has to disintegrate first, unless the tablet is designed for quick surface erosion. The definition of “complete disintegration” in the U.S. Pharmacopoeia is “that state in which any residue of the unit, except fragments of insoluble coating or capsule shell, remaining on the screen of the test apparatus is a soft mass having no palpably firm core.” Although the concept has been introduced since 1879, the mechanism of the disintegration phenomenon is not yet fully understood. The materials used as disintegrants include starches, agar, amylose, cellulose and its derivatives, gum and its derivatives, gelatin, resins, and silicone compounds.

A few mechanisms of action of disintegrants have been proposed (14). The first mechanism is evolution of gas from an effervescent couple, e.g., sodium bicarbonate with citric acid upon absorption of water. The expansion of gas can be enough to cause the tablet to disintegrate. Another mechanism is swelling of disintegrants by absorbing water to break up the tablet structure. In the tablet disintegration process, several factors may affect the disintegration. They include the rate of water absorption, porosity of the tablet, processing parameters, and effect of active ingredients, surfactants, binders, and lubricants. A general mechanism of tablet disintegration is difficult to make because tablet disintegration is a complex phenomenon (14). Fast disintegration always requires fast absorption of water into the center of the tablet. Thus, having open pore structures inside the tablets is very important for making fast dissolving tablets.

D. Water Absorption

Absorption of water into the tablet is important for binding between granules through liquid bridges and for disintegration of the tablet through swelling or dissolution of excipients. The affinity of a substance to water in its vapor state is generally referred to as hygroscopicity. Since adsorption or condensation of moisture is not an entropically favored process, the water-solid interaction must provide a sufficient enthalpic driving force for adsorption of water to occur. For water-soluble substances, dissolution of the molecules at solid surfaces can occur once multilayer condensation is established. Beyond some critical RH (RHₒ) characteristic of the solid, the condensate possesses the character of bulk solution. The water activity is depressed due to the presence of the solute, and the phenomenon of deliquesence is triggered. Substances are most often classified as hygroscopic when their RHₒ falls below the normal ambient RH. When the water vapor is adsorbed onto the solid surface of a particle, water molecules form a film on the surface. If the water vapor pressure (P_{c}) is above the pressure (Pₛ) associated with RH_{o,} further adsorption occurs and the thickness of the condensate film grows spontaneously. Under this condition, solids continue to dissolve to maintain the surface pressure at Pₛ. To reach equilibrium with the atmosphere at P_{c}, solids dissolve completely, and some degree of solution dilution must occur (15).

E. Prior Approaches of Making Fast Dissolving Tablets Employing Mannose

There are only a limited number of patent references dealing with the use of mannose in making fast disintegrating tablets. For instance, fast disintegrating tablets are proposed by sintering a preformulation product (which is a mixture of active agent, binding agent, bulking agent and/or structural agent and/or solvent) (16) [U.S. Pat. No. 6,284,270 to Lagoviyer et al.]. Sintering is a process of melting a binding agent and then resolidifying it at temperatures of 50-100° C. In this reference, mannose is listed only as a bulking agent and is not indicated for making mechanically strong tablets.

U.S. Pat. No. 6,316,029 to Jain et al. proposes use of a poorly soluble active ingredient in a nanoparticulate form for making a fast disintegrating tablet formulation (17). This reference, however, did not describe how the unique fast disintegrating tablets are prepared—it simply mentions that the nanoparticulate dispersion is combined with pharmaceutically acceptable water-soluble or water-dispersible excipient (including mannose) and the mixture is then directly compressed into tablets. No description of disintegration rate and mechanical strength of the prepared tablets is provided.

Eoga et al. propose fast-melt tablets prepared by compressing granules mixed with excipients into tablets using conventional tablet-making machinery (18) [U.S. Pat. No. 5,939,091]. Since the granules can be combined with a variety of carriers including low-density, high-moldability saccharides, low-moldability saccharides, and polyol combinations, the compressed tablets are said to exhibit an improved dissolution and disintegration profile. In this reference, mannose is merely used as filler for making tablets.

In patents dealing with shear-form matrices or flosses, monosaccharides (including mannose) are used to make shear-form matrices or flosses (similar to sugar cotton ball) that provide self-binding flowable formulations for tableting. The partially recrystallized shear-form matrices are combined with an additive to form flowable, compactible particulate blends, and these blends are either compacted at relatively low pressures or molded to produce dosage forms (19) [U.S. Pat. No. 5,840,334 to Raiden et al.].

Dobetti (20) [U.S. Pat. NO. 6,596,311] proposes yet another fast disintegrating tablet in which the formulation includes a drug in a multiparticulate form, one or more water insoluble inorganic excipients, one or more disintegrants, and optionally one or more substantially water soluble excipients. This reference does not mention mannose, and furthermore, only 4-16% by weight of water-soluble excipients is in the formulation. The water-soluble excipients are optional and not an essential part in the formulation.

As described above, only a few references mention the use of mannose in the formulation of fast dissolving tablets and, in all of them, mannose is proposed merely as a filler or a bulking agent, just like any other sugars or sugar alcohols, without having any special functions.

### SUMMARY OF THE INVENTION

The present invention employs mannose as a principal component in the fabrication of fast dissolving tablets. Mannose plays dual roles in the present invention by providing a structure-forming property and a fast-dissolving property. The structure-forming property of mannose comes from the fact that mannose powders have high compatibility and the compressed tablets can be strengthened by transient moisture treatment leading to bridging of mannose particles. The fast disintegrating as well as fast dissolving property of mannose results from an intrinsic high porosity of mannose powders and the high water-solubility of mannose.

Surprisingly, it is found that mannose-based compressed tablets are strong enough to be handled without breaking and the tablets also dissolve fast in the mouth. More surprisingly, it is observed that transient moisture treatment of mannose-based tablets results in very strong tablets that disintegrate and dissolve fast in the presence of a minimal amount of water, e.g., saliva in the mouth. This invention utilizes the high water solubility of mannose for the first time to form liquid bridges between mannose particles by transient moisture treatment at certain relative humidity values.

Accordingly, one aspect of the present invention is for a fast-disintegrating and fast-dissolving pharmaceutical tablet that also has high mechanical strength. Such a tablet comprises mannose in an amount sufficient to be principally responsible for effecting such disintegration/dissolution and mechanical strength. The tablet can be used a “sugar pill” or can also comprise a pharmaceutically effective amount of a drug of choice. It is anticipated that the pharmaceutical tablet is suitably and preferentially administered orally.

As discussed herein, tablet compression resulting in increased hardness and mechanical strength has a tendency to close pore structures and increase the time for disintegration and/or dissolution of the tablet. However, according to the principles and methods of preparation of the present invention, a tablet of the invention can have a hardness of at least 2.0 kiloponds (KP), which is roughly 20 newtons [1 KP=1 Kgf=9.807 N (newtons)], as conventionally measured. At the same time, such tablet completely disintegrates and/or dissolves within 30 seconds when covered (soaked) with water. More preferably, a tablet of the invention has a hardness of at least about 3.0 KP (ca 30 N) and dissolves in less than about 20 seconds. Most preferably, the tablet hardness is at least about 4.0 KP (ca 40 N). Some exemplary tablets prepared according to the present invention are listed in Table 3 hereinafter.

As used herein, the terms “disintegration-effective amount” and “dissolution-effective amount” are used interchangeably, and refer to the wt % amount of mannose provided in a tablet, which can effect “complete disintegration” as defined by the U.S. Pharmacopeia, as discussed hereinabove. Thus, in a preferred embodiment, a dissolution-effective amount of mannose effects complete disintegration of a tablet within 30 seconds. The wt % of mannose in such a tablet can be in the range 1-100%, and is more preferably in the range 1-99%, such as when a drug of choice is present. Some exemplary formulations are presented in Table 4 hereinafter.

Another aspect of the invention is for a method of making a pharmaceutical tablet having fast-dissolving properties. Such method comprises: (i) combining mannose and a drug compound to form an admixture thereof; (ii) compressing the admixture; (iii) subjecting the compressed admixture to relative humidity conditions effective to form liquid bridges between tablet particles; and (iv) drying the humidified, compressed product to convert the liquid bridges to solid bridges, thereby forming the desired tablet. Preferred humidity conditions are 50% RH or higher, preferably at least 70% RH, and the humidity can be provided throughout the tablet preparation process or as a separate step following compression. Additional diluents, binders, flavors, lubricants, and the like, can be incorporated into the tablet admixture as desired. Without wishing to be bound by any particular theory, the formation of “liquid bridges” and “solid bridges” may be viewed phenomenologically in terms of accounting for an observed increased adherence between tablet particles following exposure to water vapor and subsequent drying.

### DESCRIPTION OF THE DRAWINGS

FIG. 1 depicts moisture absorption of mannose powders and tablets at 75% RH and subsequent desorption by drying at 15% RH (except for tablets compressed at 300 lbs which were dried at 65% RH).

FIG. 2 depicts moisture absorption of mannose tablets at different relative humidities and subsequent drying at 15% RH (except tablets compressed at 300 lbs which were dried at 65% RH).

FIG. 3 depicts a device for disintegration testing of fast dissolving tablets as described herein.

### DESCRIPTION OF THE INVENTION

A. Survey of Materials Suitable for Making Fast Dissolving Tablets

Tablets are most easily made by compaction methods. Therefore, it is a goal of this work to develop a new method of making fast dissolving tablets by compaction. To achieve fast disintegration of the compressed tablets, water should penetrate into the tablet core as soon as possible. This requires that materials should have high wettability, and the tablet structure should also have an open porous network. Since the strength of a tablet is inversely related to porosity, it is important to find the porosity that allows fast water absorption as well as maintains high mechanical strength.

A variety of candidate materials were used to make compressed tablets, and the tablet properties, such as strength and disintegration time, were examined. Particles of each material were compressed under different pressure by a Carver press. The hardness of the tablet was tested by hand for initial qualitative measurement and also by dropping the tablet from a certain height onto the laboratory bench top or to the floor. The tablet strength was graded by assigning a number ranging from 1-5. Grade 1 indicates that the tablets are very weak and Grade 5 indicates that the tablets are very strong and comparable to regular compaction tablets. Each tablet was placed into 5 mL water and observed for 30 seconds to see if it disintegrated and dissolved in that time period. The results obtained for tablets made from various materials are listed in Table 1.

TABLE 1
Strength and Disintegration Properties of tablets made of various materials.

| | | | | |
|---|---|---|---|---|
| | | Compression | | |
| | | Pressure | Tablet | |
| Material | Appearance | (MPa) | Strength | Disintegration Characteristics |

Carbohydrates

| | | | | |
|---|---|---|---|---|
| Sucrose | Large square | 70.2 | 2 | No disintegration of the tablet. |
| | crystals | | | Formation of a viscous layer on the |
| | | | | surface of the tablet. |
| | | | | Slow dissolution of the tablet. |
| | | 91.3 | 2 | No improvement. |
| Maltose | White powder | 70.2 | 3 | Total disintegration of the entire tablet. |
| Glucose | White powder | 70.2 | 2 | Good water penetration. |
| | | | | Appearance of bubbles from the tablet. |
| | | | | No disintegration. |
| Mannitol | White powder | 70.2 | 2 | Slow disintegration at the inner part |
| | | | | of the tablet. |
| Lactose | White powder | 70.2 | 3 | Disintegration of the outer part of |
| monohydrate | | | | the tablet |
| | | | | Wetting but no dissolution of the |
| | | | | inner part of the tablet. |
| Glycine | White powder | 105 | 2 | Appearance of bubbles from the tablet. |
| | | | | Quick disintegration. |
| Glutamic acid | White powder | 105 | 3 | Appearance of bubbles from the tablet. |
| | | | | Quick disintegration. |
| Mannose | Yellow powder | 35.1 | 4 | Good mechanical strength. |
| | | | | Fast disintegration and dissolution. |
| | | 70.2 | 4 | Disintegration of the outer part, and |
| | | | | dissolution of the inner part of the |
| | | | | tablet. |
| | | 105 | 4 | Dissolution of the most part of the |
| | | | | tablet without disintegration. |

Polymers

| | | | | |
|---|---|---|---|---|
| Carrageenan + | Yellow powder | 70.2 | 3 | Swelling of the outer portion of the |
| gelatin | | | | tablet to form a viscous layer. |
| | | | | Dry inner core without disintegration. |
| Agar | Yellow powders | 70.2 | 4 | Instant absorption of water and |
| | | | | disintegration. |
| Dextrin | Flake like | 70.2 | 5 | Formation of a viscous layer without |
| | sheet | | | disintegration of the tablet. |
| Pectin | Yellow powder | 70.2 | 4 | Formation of a viscous layer without |
| | | | | disintegration of the tablet. |
| Cellulose | White powder | 70.2 | 5 | No disintegration. |
| acetate | | | | |
| PVA | White powder | 70.2 | 5 | No disintegration. |
| PVP | White powder | 70.2 | 5 | No water penetration. |
| Cross linked | White powder | 70.2 | 4 | Formation of a viscous layer without |
| PVP | | | | disintegration of the tablet. |
| | | | | Presence of a hard dry core even |
| | | | | after 1 minute. |
| Amylose | White powder | 35.1 | 2 | Immediate formation of bubbles. |
| | | | | Immediate disintegration of the |
| | | | | tablet but maintenance of the overall |
| | | | | tablet shape. |
| | | 70.2 | 2 | Weak tablet. |
| | | | | Quick disintegration. |
| | | 105 | 3 | Weak tablet. |
| | | | | Quick disintegration. |
| Chitosan | Very thick | 70.2 | 2 | Poor wettability. |
| | flakes | | | No disintegration. |
| Guar Gum | Yellow powder | 70.2 | 2 | Slow absorption of water to form a |
| | | | | viscous layer on the surface. |
| | | 105 | 2 | No disintegration. |
| Beta- | White powder | 105 | 4 | No disintegration. |
| Cyclodextrin | | | | |
| | | 35.1 | 4 | No disintegration. |
| Dextrin | White powder | 70.2 | 4 | No disintegration. |
| Molecular | White powder | 105 | 4 | Formation of large bubbles. |
| sieve | | | | Breaking of the tablet into chips. |
| Potato starch | White powder | 70.2 | 3 | Swelling and softening of the tablet. |
| Micro | White powder | 35.1 | 5 | Breaking of the tablet into big pieces |
| crystalline | | | | and then into smaller pieces. |
| cellulose | | 105 | 5 | Breaking of the tablet into chips and |
| | | | | then slowly into powder. |
| Ac-Di-Sol | White powder | 35.1 | 2 | Swelling of the outer layer. |
| | | | | Dry inner core. |
| | | 70.2 | 3 | Swelling of the thin outer layer. |
| | | 105 | 4 | Delayed swelling of the surface. |

Organic salts

| | | | | |
|---|---|---|---|---|
| Calcium | White powder | 105 | 4 | No disintegration. |
| Acetate | | | | |
| Zinc Acetate | White powder | 70.2 | 3 | No disintegration. |
| Calcium | White powder | 35.1 | 4 | Immediate formation of large bubbles. |
| Gluconate | | | | Formation of soft, viscous outer |
| | | | | layer with hard inner core. |
| Salicylic Acid | White powder | 35.1 | 1 | Floating of the tablet. |
| | | | | No disintegration. |
| | | 70.2 | 2 | No disintegration. |
| | | 105 | 2 | No disintegration. |
| Sodium | White powder | 35.1 | 1 | Instant disintegration |
| Saccharin | | 70.2 | 2 | Instant disintegration |
| | | 105 | 2 | Instant disintegration |

Inorganic materials

| | | | | |
|---|---|---|---|---|
| Sodium | White powder | 54.8 | 4 | No disintegration. |
| Carbonate | | | | Generation of carbon dioxide in acid |
| | | | | without disintegration. |
| | | 91.3 | 3 | No disintegration. |
| Fumed silica | Very fluffy | 70.2 | 2 | No wetting. |
| | insoluble powder | | | Floating of the tablet. |
| Sodium | Square crystals | 70.2 | 2 | No disintegration. |
| Chloride | | 105 | 2 | No disintegration. |
| Calcium | White powder | 105 | 3 | Immediate formation of some bubbles. |
| Phosphate, | | | | Minor collapse on the tablet without |
| Monobasic | | | | major disintegration. |
| Iron | Black powder | 105 | 2 | No disintegration. |
| Calcium | White powder | 105 | 4 | Immediate formation of bubbles. |
| Chloride | | | | No disintegration. |
| Calcium | White powder | 105 | 2 | Instant disintegration without |
| Carbonate | | | | swelling. |
| | | | | Maintenance of the overall tablet |
| | | | | form. |
| Aluminum | White powder | 105 | 4 | No disintegration. |
| Hydroxide | | | | Limited water penetration. |
| DITAB | White powder | 70.2 | 4 | Immediate formation of bubbles. |
| (Calcium | | | | Immediate disintegration and very |
| Phosphate, | | | | fast collapse of the tablet. |
| Dibasic) | | | | |
| Talc | White powder | 35.1 | 2 | No disintegration. |
| | | 70.2 | 2 | No disintegration. |
| | | 105 | 3 | No disintegration. |

Drugs

| | | | | |
|---|---|---|---|---|
| Ibuprofen | White powder | 70.2 | 3 | No wetting. |
| | | | | Floating of the tablet. |
| Aspirin | White powder | 35.1 | 2 | Immediate formation of bubbles. |
| | | | | No further disintegration after 20 sec |
| | | | | with the half of the tablet intact. |
| | | 70.2 | 3 | No disintegration for the first 30 sec, |
| | | | | followed by little disintegration. |
| | | 105 | 4 | No disintegration for the first 30 sec, |
| | | | | followed by slow disintegration. |
| Caffeine | White powder | 35.1 | 3 | Immediate release of particles from |
| | | | | the tablet surface. |
| | | | | Collapse of the entire tablet. |
| | | 70.2 | 4 | Breaking into particles after 20 sec. |
| Theophyline | White powder | 35.1 | | No disintegration. |
| | | 70.2 | | No disintegration. |
| | | 105 | | No disintegration. |

Since the first criterion for making fast dissolving tablets is fast disintegration, those materials that resulted in fast disintegration were screened. They were maltose, glycine, glutamic acid, mannose, agar, amylose, sodium saccharin, calcium carbonate, and DITAB (calcium phosphate). Since the second criterion is high mechanical strength, those with tablet strength of Grade 4 or above were screened. Thus, the remaining materials were mannose, agar, and DITAB. DITAB could form strong tablets at low pressures and the tablets could be easily disintegrated. However, DITAB is not soluble in water and thus it may not provide a good mouth feeling. Agar was also a good candidate for making tablets with fast disintegrating property. Agar particles, however, do not dissolve (or soluble) due to the hydrogel nature by physical crosslinking of agar molecules. Both DITAB and agar particles will be useful in making fast disintegrating tablets as long as the disintegrated particles do not cause any undesirable feelings in the mouth. Mannose can form tablets with low friability even at relative low pressures, and the tablets can disintegrate very fast. Furthermore, due to its high water solubility, the disintegrated particles dissolve completely very quickly. The three materials (mannose, DITAB, and agar) can be used separately as well as in combinations for making fast dissolving tablets.

B. Property of Mannose

D-Mannose is a simple sugar, which is a stereoisomer of glucose. Mannose is a naturally occurring plant-based sugar (a nutritional food substance) found in cranberry and pineapple juice. It is also produced in the body and has no toxicity. When D-mannose is ingested into the body, most of it is rapidly absorbed through the stomach and upper GI tract before reaching the intestines, emptying into the urine through the kidneys. Mannose is not metabolized in the body and thus it does not interfere with blood glucose regulation, even for diabetics. It is known to be safe for pregnant women and children.

Mannose is a white crystalline powder with melting temperature of 132° C. One gram of mannose can dissolve in 0.4 mL of water (21). This material has been listed in the Inactive Ingredient guide published by FDA. Mannose powder can be purchased from various sources, including Amresco, Inc. The powder is in a crystalline form as confirmed by x-ray powder diffraction (XPRD) analysis. No significant changes in the XPRD patterns are observed upon compression indicating that the crystal structure of mannose is maintained after compression.

The structures of mannose powders were examined by SEM, which revealed that they have a highly porous structure. This may help generate large surface areas when they are compressed into tablets. It is understandable that the pores between crystals allow fast absorption of water by capillary force. The particles are also spherical for good flowability.

An isothermal moisture absorption test was conducted to examine moisture absorption onto the mannose powders. The moisture absorption property is important for finding the optimal condition for formation of liquid bridging as well as the drying process. As observed, the moisture absorption onto mannose powders is almost negligible up to 70% relative humidity (% RH) at 25° C. Mannose picks up water at 70% RH and water absorption increases linearly as the RH increases. Thus, 70% RH is the critical relative humidity for mannose.

C. Mechanism of Fast Disintegration of the Mannose Tablets

It was observed that water penetration into mannose tablets was instantaneous. Unless the porosity of the tablet was too low to have effective water penetration, the void space inside the tablet was quickly filled with water. However, water penetration alone cannot explain the fast disintegration property of mannose tablets. Some tablets with intermediate pore size with quick water absorption still did not result in fast disintegration. One possible explanation for fast disintegration of mannose tablets is that mannose can quickly dissolve inside the tablet due to its high aqueous solubility to weaken the whole structure of the tablet. The tablet cannot withstand its structure and will collapse to result in disintegration. To test this hypothesis, disintegration tests of mannose tablets in different media were carried out. The mannose tablets that were compressed at 300 lbs with 0.5 inch punches were used as sample tablets for the disintegration test. The experimental conditions were kept the same except for the disintegration media. The disintegration media consisted of different concentrations of mannose solutions and other solvents.

Results of the disintegration test are summarized in Table 2. It was observed that the time for the media penetrating into the tablet is negligible compared with the whole disintegration time in every case. The disintegration time increased as the mannose concentration increased. Solubilities of mannose in pyridine and ethanol are 0.29 g/mL and 0.004 g/mL, respectively. The Noyes and Whitney equation suggests that both the concentration of solution and the solubility of the material affect the dissolution process. Therefore, it appears that the dissolution rate of the material determines the disintegration kinetics of the tablets.

TABLE 2
Disintegration times of mannose tablets in different disintegration media.

| | | Mannose | Mannose | Mannose | Mannose | Mannose | | |
|---|---|---|---|---|---|---|---|---|
| | Pure | solution | solution | solution | solution | solution | | |
| | water | 0.1 g/mL | 0.2 g/mL | 0.4 g/mL | 0.6 g/mL | 2.5 g/mL | Pyridine | Ethanol |
| Disintegration | 7.0 ± 0.3 | 11.1 ± 2.1 | 14.0 ± 2.3 | 18.7 ± 1.6 | 36.9 ± 16.3 | >7,200 | 532 ± 50 | >7,200 |
| time (Sec) | | | | | | | | |

D. Mechanism of Increased Strength of Mannose Tablets by Moisture Treatment

The hardness of the tablets before the moisture treatment was only 0.3 kiloponds (KP) but it increased to 4.3 KP after moisture treatment. This strength is close to many ordinary tablets prepared by compaction without any fast dissolving properties. The substantial increase in mechanical strength of the mannose tablets is due to formation of liquid bridges in the presence of moisture that in turn become solid bridges after drying.

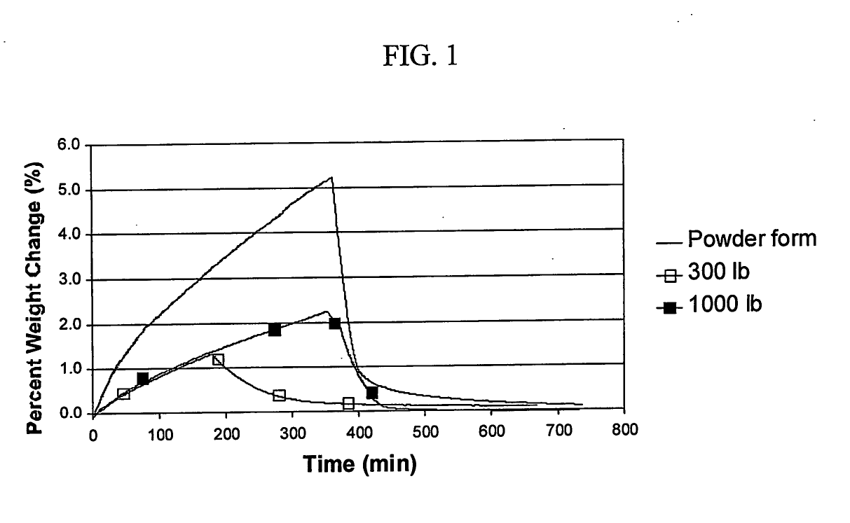

The moisture absorption rates of tablets with different porosity were compared. The moisture sorption tests were conducted in the symmetric vapor sorption analyzer SGA-10. Mannose powder (300 mg) or tablets of the same weight but varying in porosity were tested. Different porosities were achieved by compressing the tablets with 300 lbs or 1000 lbs. The tablets were first dried and then the humidity was kept at 75% RH; at the end of the sorption period, the humidity dropped to 15% RH for drying (except for tablets compressed at 300 lbs which were dried at 65% RH). As seen in FIG. 1, the moisture absorption increased linearly with time. The moisture absorption on mannose powders was faster than that on mannose tablets, and this is easily understandable. Although the tablets prepared under different compression pressure had significantly different porosities, the moisture sorption rate was almost the same. As the powders and tablets were moved into the 15% RH chamber, desorption of water occurred faster than absorption.

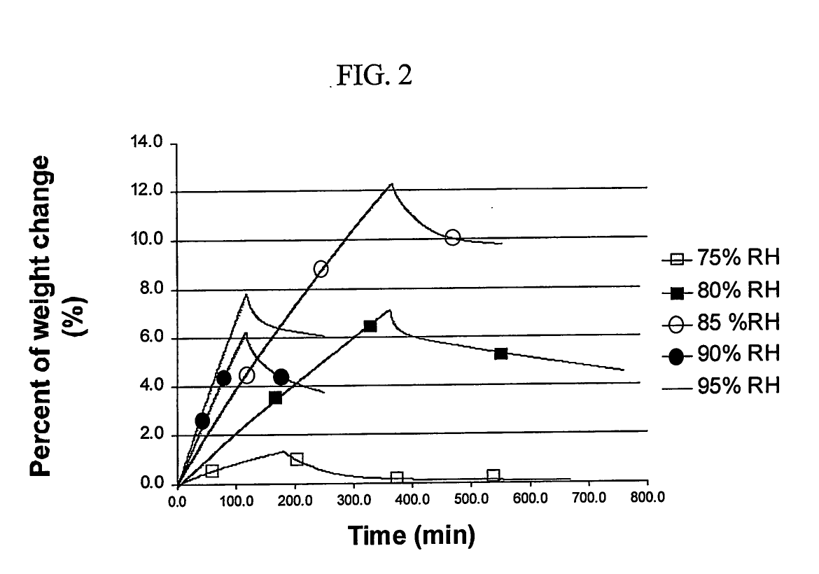

The same type of tablets compressed at 300 lbs were tested for their moisture uptake at different relative humidities. As shown in FIG. 2, the rate of moisture absorption increased as the relative humidity increased from 75% to 95%. Comparison of the drying parts of the moisture curve in both FIGS 1 and 2 indicates that the water absorbed into the mannose tablets can be evaporated rather quickly if the absorbed water is less than 6%. Upon exposure to 15% RH, all absorbed water is removed from the mannose tablets (see moisture absorption curve at 75% RH in FIG. 2). On the other hand, when moisture absorption exceeds 6%, the subsequent drying step is very slow. Tablets removed from the moisture chamber were overly shrunken or even distorted. This indicates the effect of moisture on tablet property changed drastically when the moisture content exceeds 6%. Thus, it is important to moisturize for liquid bridge formation at 75% RH.

To test the effects of porosity and duration of moisture treatment on the properties of the resultant tablets, a series of tablets were prepared. As shown in Table 3, the compression forces ranged from 100 to 1000 lbs. The tablets were placed in a 75% RH humidity chamber. The tablets were sampled at 3, 4, 6 and 8 hours, and strength was tested by a hardness tester. The disintegration tests were performed by the method described above and the thickness and diameter of the tablets were measured before and after the treatment to calculate the degree of volume reduction.

TABLE 3
Properties of tablets made at different compression pressure
and with different duration of moisture treatment.

| Time of | | | | |
|---|---|---|---|---|
| moisture | | | Disinte- | Volume |
| treatment | Compression | Hardness | gration | reduc- |
| (hours) | force (lbs) | (KP) | time (sec) | tion (%) |
| 0 | 100 | 1.5 ± 0.2 | 5.1 ± 1.4 | |
| | 300 | 2.5 ± 0.3 | 7.7 ± 1.5 | |
| | 600 | 4.2 ± 0.1 | 10.4 ± 0.2 | |
| | 1,000 | 4.6 ± 0.6 | 15.2 ± 3.2 | |
| 3 | 100 | 2.07 ± 0.3 | 5.6 ± 0.7 | 4.1 ± 2.2 |
| | 300 | 4.7 ± 0.6 | 13 ± 0.2 | 5.7 ± 1.1 |
| | 600 | 5.7 ± 1.7 | 22.7 ± 1.4 | 6.1 ± 1.0 |
| | 1,000 | 6.1 ± 0.6 | 35.1 ± 1.1 | 5.2 ± 0.7 |
| 4 | 100 | 2.3 ± 0.6 | 5.4 ± 0.8 | 4.5 ± 0.8 |
| | 300 | 4.9 ± 0.8 | 11.4 ± 0.9 | 5.9 ± 1.3 |
| | 600 | 5.1 ± 0.7 | 23.5 ± 2.5 | 6.9 ± 1.3 |
| | 1,000 | 6.7 ± 0.6 | 47.1 ± 5.5 | 6.2 ± 1.3 |
| 6 | 100 | 2.8 ± 0.8 | 5.5 ± 0.9 | 5.6 ± 3.8 |
| | 300 | 4.1 ± 0.3 | 13 ± 0.4 | 5.7 ± 0.8 |
| | 600 | 5.1 ± 0.9 | 32 ± 6 | 7.3 ± 0.9 |
| | 1,000 | 6.5 ± 1 | 38 ± 1.6 | 7.4 ± 1.3 |
| 8 | 100 | 1.2 | 5.3 | 10.4 |
| | 300 | 5.6 ± 0.1 | 15.6 ± 0.3 | 7.1 ± 1.5 |
| | 600 | 5.4 ± 0.5 | 35.5 ± 4.7 | 8.7 ± 1.7 |
| | 1,000 | 6.1 ± 0.6 | 42.8 ± 2.4 | 8.4 ± 1.1 |

As summarized in Table 3, the tablet strength improved after treatment. The strength gained by a tablet compressed at 100 lbs was not enough in all cases. On the contrary, the tablets compressed at 1000 lbs gained some strength but their disintegration times were significantly increased. The tablet compressed at 300 lbs gained strength gradually with moisture treatment time but the disintegration time remained in essentially the same range. These different responses to moisture treatment may indicate differences in the pore size distribution inside the tablet, especially the lower portion of the size distribution. The tablets compressed at 100 lbs have less pores with size small enough for the moisture layers to merge together to make liquid bridges. On the other hand, tablets compressed by 1000 lbs have many small pores. With excess amount of small pores and lack of large pores, the tablet porosity and surface area inside the tablet significantly decreased, and these will lead to slow disintegration. This point is also supported by volume reduction data. With about the same amount of water absorbed, the volume reductions tend to increase with increase in compression. Therefore, it is very important to find an optimized pore distribution such that it gives enough pores to merge to gain strength and in the meantime contain a large enough portion of pores for disintegration.

Additional visual confirmation of the tablet structural changes was made from SEM pictures. The picture of a tablet compressed at 300 lbs without humidity treatment shows that some individual particles are still not merged together. However, after the humidity treatment small pores were significantly decreased or merged, but larger pores were still intact. The tablet compressed at 1000 lbs after humidity treatment showed many merged pores with less large pores. This visual examination supports the data of the hardness and disintegration tests.

E. Preparation of Fast Dissolving Tablets Using Mannose

This invention is about using mannose as the main component in making fast dissolving tablets. The mannose particles are usually in the form of a porous aggregate with a high surface area The high water-solubility and porous particle structure of mannose are exploited in this invention to make fast dissolving tablets.

In this invention, mannose is incorporated into a fast dissolving tablet in such a way that when the tablet is exposed to water, water is taken up immediately into the tablet to dissolve mannose particles first throughout the tablet. The other excipient particles either near mannose particles or bridged by mannose particles lost bonding, and as a result the tablet becomes disintegrated. Mannose particles can be incorporated with other materials by either mixing or granulating with them.

The strength of tablets containing mannose can be enhanced by the process of moisture absorption and subsequent drying. The mannose has a critical relative humidity of 70% at 25° C. When the relative humidity is beyond this point, mannose absorbs water from the environment, leading to formation of a liquid layer on the surface of mannose particles. As the liquid layer grows, nearby liquid layers merge together to form liquid bridges between particles. Upon drying those liquid bridges, solid bridges form to make stable bonds between particles. These bonds significantly increase the tablet strength. By optimizing the amount of water absorbed and the formation of liquid bridges, the strength of the tablets can be controlled. This method does not significantly reduce the tablet volume and thus the pores inside the tablet are maintained for fast disintegration and dissolution.

Mannose-Based Fast Melting Tablets

A. Formulations

Mannose can be mixed with an active ingredient and other excipients. The amount of mannose in the formulation can range from 1% to 99%. The active ingredients include drugs and nutritional components. The active ingredients can be coated for taste masking, if necessary. The excipients include pharmaceutically acceptable diluents, binders, disintegrants, sweeteners, flavor agents, coloring agents, and lubricants.

For diluents, virtually all pharmaceutically suitable diluents can be used together with mannose. It is preferred to choose from water-soluble sugar or sugar alcohols. Examples are dextrose, fructose, lactitol, lactose, maltitol, maltose, mannitol, sorbitol, sucrose, erythritol, trehalose, dextrates and xylitol. One or more highly water-soluble polymers can be also chosen. Examples include polysaccharides, such as dextrin, maltodextrin polydextose, povidone, polyethylene glycol, polyethylene oxide, polyvinyl alcohol, hydroxyethyl cellulose, hydroxyethylmethyl cellulose, hydroxypropyl cellulose, and gelatin. Highly water-soluble organic acids and salts and inorganic salts can also be used. Examples include citric acid, potassium bicarbonate, potassium chloride, potassium citrate, sodium bicarbonate, sodium chloride, sodium citrate, sodium phosphate dibasic, and sodium phosphate monobasic.

Diluents that are less water-soluble but highly dispersible can also be used. They include agar, kaolin, starch, pregelatinized starch, microcrystalline cellulose, silicified microcrystalline cellulose, powdered cellulose, cyclodextrins, ethylcellulose, chitosan, cellulose acetate, calcium sulfate, calcium carbonate, dibasic calcium phosphate, tribasic calcium phosphate, and carboxymethylcellulose-calcium salt. Materials of various combinations of carbohydrates and polymers can also be used. Examples are STARLAC (spray-dried solid containing 15% maize starch and 85% alpha-lactose monohydrate from Roquette American, Inc.), MICROCELAC (spray-dried solid containing 75% alpha-lactose monohydrate and 25% microcrystalline cellulose from Meggle Excipients & Technology), and CELLACTOSE (spray-dried compound consisting of 75% alpha-lactose monohydrate and 25% cellulose powder by Meggle). The preferred grade of the material used as bulk diluents is the direct compression grade. Materials prepared with high porosity, e.g., by spray drying, are even more preferred. Examples of porous bulk diluents are STARLAC, MICROCELAC, CELLACTOSE, and MANNOGEM EZ spray (spray-dried mannitol from SPI Pharma. Inc.), and combinations thereof.

In addition to mannose, some other carbohydrates that have low critical relative humidity can be added to help moisture sorption during humidification. Examples of these carbohydrates are fructose, dextrates, dextrose, sorbitol and xylitol. If polymers or other excipients used in the formulation can absorb moisture above the relative humidity used for moisture treatment, they can also help strengthen the tablet during humidification.

Exemplary disintegrants include starches, crosslinked polyvinylpyrrolidone, croscarmellose sodium, sodium starch glycolate, and superporous hydrogel. Sweeteners include natural and artificial sweeteners, such as sodium saccharin, aspartame, and cyclamate. Examples of flavor agents include fruit flavor, bubble gum flavor, and the like. Suitable coloring agents include food dyes, food lake dye, and the like. Acceptable lubricants include magnesium stearate, stearic acid, polyethylene glycol, talc, sodium stearyl fumarate, colloidal silicon dioxide, glyceryl behenate, and the like.

B. Processing

The process of making fast dissolving tablets according to the present invention includes mixing of the components, an optional granulation step, compression of the mixed components or granules, transient treatment of the formed tablets with moisture in the humidity chamber (i.e., humidity treatment), and drying of the tablets.

The components of a fast dissolving tablet formulation are mixed together. This mixture can be used for direct compression or it can be granulated first for better flowability and/or compressibility. Granulation of the components can be achieved by wet granulation using low shear granulators, high shear granulators, or fluid bed granulators. Granulation is conducted at a relative humidity above the critical relative humidity of mannose. In the absence of a binder solution, mannose powders absorb water throughout the process and form liquid bridges between particles to achieve wet granulation. By using this method, uneven distribution of water in the mixture is greatly reduced throughout the granulation process. In this way, water is mainly distributed at the surface of the mannose particles to form liquid bridges between particles without excessively dissolving mannose particles.

The mixture or the granules are then compressed into tablets. The compression force is kept low so that most pores inside the tablet can be maintained. The compression force typically ranges from 1 MPa to 50 MPa.

The prepared tablets are transferred to a device for humidity treatment. The device can be a chamber, an oven, or simply a whole room. The humidity of this device is raised above the critical relative humidity of mannose, which is 70%. The rate of water absorption by mannose varies according to the relative humidity. The higher the relative humidity, the faster is the moisture sorption. The relative humidity can be kept constant or varied if necessary. The time for humidification can be adjusted depending on the amount of mannose in the tablet and relative humidity in the device. The time can range from 10 min to 24 hours. The temperature in the humidification step can also range from room temperature to 60° C.

After the humidity treatment, the tablets are dried. Drying methods can be air drying, vacuumed drying, oven drying, microwave drying or other suitable methods. The tablet can be dried at room temperature or in the oven with a temperature less than 100° C. The humidification and drying process can be repeated several times if necessary.

C. Disintegration Test

Fast dissolving tablets are supposed to disintegrate in the mouth by saliva. Saliva in the mouth is limited, and no simulated saliva test solution is found in United States Pharmacopeia The following two methods were used to evaluate the disintegration and dissolution of the fast dissolving tablets.

Disintegration Test A:

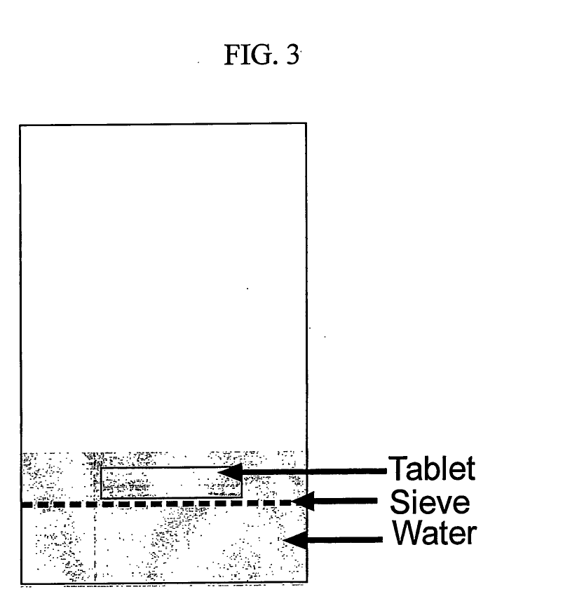

The disintegration times for the tablets were measured using the device shown in FIG. 3. Fast disintegration tablets are supposed to disintegrate in the mouth by saliva. The amount of saliva in the mouth is limited, and consequently the disintegration test should reflect the application condition. A new device was designed to test the disintegration time. A 10-mesh sieve was put into a cylinder and 2 mL of water was filled between the bottom and the sieve. An additional 1 mL of water was poured into the device so that there was 2mL of water below the sieve and 1 mL of water above the sieve. The device was put on a shaker in a 37° C. water bath. When the tablet was immersed into water, the shaker provided 150 rpm horizontal back and forth motions. The time taken for all parts of the tablet to go through the sieve was recorded as the disintegration time.

The disintegration time for mannose tablets was observed to be 7.5 seconds. The complete dissolution time was longer than the disintegration time, but the disintegration time provided a good estimate of the actual breaking of the tablets into smaller pieces in the mouth.

Disintegration Test B:

This method is a modified version of the method developed by Dor and Fix (22). The method utilized a texture analyzer (TA XT2®, Texture Technologies Corp; Scarsdale, N.Y.). A tablet was adhered to the bottom of a probe, which is attached to the load cell with a very thin layer of glue or a double-sided copper tape. With constant force, the tablet was approached to a filter paper soaked with water, which was connected to a water reservoir. When the tablet started to disintegrate, the rate of movement that the probe travels showed a sudden increase. This increased rate continued until the tablet was disintegrated. The time that the increased rate of movement stopped was taken as the disintegration time.

Disintegration Test C:

A tablet was put into the buccal cavity of healthy adult volunteers to measure the time needed for complete disintegration by saliva Three male volunteers took part in this test (average 35 years old). Each volunteer tested the same formulation at least twice and the average value was adopted.

D. Tablet Strength

Tablet strength was measured by a texture analyzer (TA XT2®, Texture Technologies Corp.; Scarsdale, N.Y.). The force that causes a diametrical failure (i.e., clear breaking) of a tablet was taken as the indicator for the tablet strength.

E. Examples of Mannose-Based Fast Dissolving Tablets

The mannose-based fast dissolving tablets were prepared as follows. For each example listed in Table 1, 300 mg of the component mixture was poured into a die of 0.5 inch in diameter and subsequently compressed by a Carver press at 300 lbs or at 600 lbs. The prepared tablets were placed in a drykeeper desiccator (Sanplatec Corp.; Osaka, Japan) with 75% RH at 25° C., which was created by placing saturated sodium chloride solution in the drykeeper desiccator. The tablets were taken out after 4 hours and air dried for 8 hours at 25° C. or at room temperature. The tablet hardness and the disintegration time were measured.

TABLE 4
Tablet hardness and disintegration time of mannose-based
fast dissolving tablets made of various compositions.

| | | | | Compression | Hardness | Disintegration | Disintegration |
|---|---|---|---|---|---|---|---|
| Example | Components in the mixture | | | pressure (lbs) | (Newtons) | time (sec) | test |
| 1 | Mannose | | | 600 | 51.5 | 15.2 | A |
| 2 | Mannose | 21 | g | 300 | 32.3 | 7.3 | A |
| | Mannogem EZ* | 9 | g | | | | |
| 3 | Mannose | 21 | g | 300 | 34.7 | 10.7 | A |
| | Mannitol | 9 | g | | | | |
| | (Mannose/mannitol | | | | | | |
| | granule)** | | | | | | |
| 4 | Mannose | 21 | g | 300 | 32.2 | 34.9 | A |
| | Xylitol | 9 | g | | | | |
| | (Mannose/xylitol | | | | | | |
| | granule)*** | | | | | | |
| 5 | Mannose | 10 | g | 300 | 17.3 | 3 | A |
| | Maltrin 250 | 6 | g | | | | |
| | StarLac | 14 | g | | | | |
| 6 | Mannose | 10 | g | 300 | 27.9 | 7 | A |
| | Sugartab | 6 | g | | | | |
| | Mannogem EZ | 14 | g | | | | |
| 7 | Mannose | 10 | g | 300 | 35.0 | 10 | A |
| | Maltrin 250 | 6 | g | | | | |
| | Mannogem EZ | 14 | g | | | | |
| 8 | Mannose | 5 | g | 300 | 36.9 | 19 | A |
| | Maltrin 250 | 7.5 | g | | | | |
| | Mannogem EZ | 17.5 | g | | | | |
| 9 | Mannoseb | 5 | g | 300 | 57.3 | 11 | A |
| | Maltrin 250 | 7.5 | g | | | | |
| | StarLac | 17.5 | g | | | | |
| 10 | Mannose | 10 | g | 300 | 15.9 | 7 | A |
| | Maltrin 150 | 6 | g | | | | |
| | StarLac | 14 | g | | | | |
| 11 | Mannose | 10 | g | 300 | 10.2 | 5 | A |
| | Maltrin 250 | 4.5 | g | | | | |
| | StarLac | 10.5 | g | | | | |
| | Ketoprofen | 5 | g | | | | |
| 12 | Mannose | 10 | g | 300 | 44.2 | 12 | A |
| | Maltrin 250 | 6 | g | | | | |
| | StarLac | 9 | g | | | | |
| | Acetaminophen | 5 | g | | | | |
| 13 | Mannose | 10 | g | 300 | 25.6 | 9.3 | A |
| | Maltrin 250 | 4.5 | g | | | | |
| | StarLac | 10.5 | g | | | | |
| | Acetaminophen | 50 | g | | | | |
| 14 | Mannose | 10 | g | 300 | 26.2 | 8.7 | A |
| | Maltrin 250 | 4.5 | g | | | | |
| | StarLac | 10.5 | g | | | | |
| | Acetaminophen | 5 | g | | | | |
| 15 | Mannose | 25 | g | 300 | 32.7 | 5 | B |
| | Ketoprofen | 5 | g | | | | |
| 16 | Mannose | 20 | g | 300 | 27.4 | 7.2 | B |
| | Acetaminophen | 10 | g | | | | |
| 17 | Mannose | 20 | g | 600 | 28.0 | 7.8 | B |
| | Aspirin | 10 | g | | | | |
| 18 | Mannose | 25 | g | 600 | 41.0 | 5 | A |
| | Acetaminophen | 5 | g | | | | |
| 19 | Mannose | 25 | g | 600 | 29.1 | 9.8 | B |
| | Ketoprofen | 5 | g | | | | |
| | SPH**** | 0.6 | g | | | | |
| 20 | Mannose | 19.52 | g | 300 | 32.3 | 7.3 | C |
| | Mannitol | 8.37 | g | | | | |
| | Loratadine | 1.00 | g | | | | |
| | Crosscamellose | 0.90 | g | | | | |
| | sodium | | | | | | |
| | Magnesium | 0.15 | g | | | | |
| | stearate | | | | | | |
| | Aspartam | 0.06 | g | | | | |
| | (Mannose/mannitol | | | | | | |
| | granule)** | | | | | | |

*Mannogem ™ EZ Spray Dried Mannitol.
**Mannose and mannitol were mixed and granulated.
***Mannose and xylitol were mixed and granulated.
****Superporous hydrogel (crosslinked poly(acrylic acid), sodium salt); See, e.g., U.S. Pat. No. 6,271,278, the pertinent disclosure of which is incorporated herein by reference.

The tablet hardness and disintegration times are the average values of three samples.

The present invention has been described hereinabove by reference to certain examples for purposes of clarity and explanation. It should be appreciated that certain obvious improvements and modifications can be practiced within the scope of the appended claims.

### REFERENCES

The pertinent portions of the following references are incorporated herein by reference.
1. Chang, R.-K., Guo, X., Burnside, B. A., and Couch, R. A. Fast-dissolving tablets. Pharmaceutical Development and Technology 24: 52-58, 2000.
2. Habib, W., Khankari, R. K., and Hontz, J. Fast-dissolve drug delivery systems. Critical Reviews in Therapeutic Drug Carrier Systems 17: 61-72, 2000.
3. Van Scoik, K. G. Solid pharmaceutical dosage in tablet triturate form and method of producing same. U.S. Pat. No. 5,082,667, 1992.
4. Roser, B. J., and Blair, J. Rapidly soluble oral solid dosage forms, methods of making same, and compositions thereof. U.S. Pat. No. 5,762,961, 1998.
5. Dobetti, L. Fast-melting tablets: Developments and technologies. Pharmaceutical Technology (North America) 44-46,48-50, 2001.
6. Cousin, G., Bruna, E., and Gendrot, E. Rapidly disintegratable multiparticular tablet. U.S. Pat. No. 5,464,632, 1995.
7. Wehling, F., Schuehle, S., and Madamala, N. Effervescent dosage form with microparticles. U.S. Pat. No. 5,178,878, 1993.
8. Amborn, J., and Tiger, V. Apparatus for handling and packaging friable tablets. U.S. Pat. No. 6,311,462, 2001.
9. Khankari, R. K., Hontz, J., Chastain, S. J., and Katzner, L. Rapidly dissolving robust dosage form. U.S. Pat. No. 6,024,981, 2000.
10. Mizumoto, T., Masuda, Y., and Fukui, M. Intrabuccally dissolving compressed moldings and production process thereof U.S. Pat. No. 5,576,014, 1996.
11. Mizumoto, T., Masuda, Y., Kajiyama, A., Yanagisawa, M., and Nyshadham, J. R. Tablets quickly disintegrating in the oral cavity and process for producing the same. U.S. Pat. No. 6,589,554, 2003.
12. Rumpf, H. Adhesion and solidity of agglomerates. Comparison between a model calculation and experiment. Pharmazeutische Industrie 34: 270-281, 1972.
13. Marshall, K. Compression and conslidation of powdered solid. In The theory and practice of industrial pharmacy, Lachman, L., Lieberman, H. A., and Kanig, J. L. (Eds.), Lea & Febiger, 1986.
14. Lowenthal, W. Disintegration of tablets. Journal of Pharmaceutical Sciences 61: 695-711, 1972.
15. Van Campen, L., Amidon, G. L., and Zografi, G. Moisture sorption kinetics for water-soluble substances. I: Theoretical considerations of heat transport control. Journal of Pharmaceutical Sciences 72: 1381-1388, 1983.
16. Lagoviyer, Y., Levinson, R. S., Stotler, D., and Riley, T. C. Means for creating a mass having structural integrity. U.S. Pat. No. 6,284,270, 2001.
17. Jain, R. A., Ruddy, S. B., Cumming, K. I., Clancy, M. J. A., and Codd, J. E. Rapidly disintegrating solid oral dosage form. U.S. Pat. No. 6,316,029, 2001.
18. Eoga, A. B. J. and Valia, K. H. Method for making fast-melt tablets. U.S. Pat. No. 5,939,091, 1999.
19. Raiden, M. G., Sanghvi, P. P., Misra, T. K., Currington, J. W., Kamath, S. V., and Pankhania, M. G. Self-binding shearform compositions. U.S. Pat. No. 5,840,334, 1998.
20. Dobetti, L. Fast disintegrating tablets. U.S. Pat. No. 6,596,311, 2003.
21. Stecher, P. G. The merk index of chemicals and drugs. 638 pp. 1960.
22. Dor, P. J. M., and Fix, J. A. In vitro determination of disintegration time of quick-dissolve tablets using a new method. Pharmaceutical Development and Technology 5: 575-577, 2000.

1. A fast-dissolving pharmaceutical tablet comprising a dissolution-effective amount of mannose, which tablet has a hardness of at least about 20 newtons and which disintegrates within about 30 seconds in the presence of an amount of water sufficient to soak the tablet.

2. The tablet of claim 1, wherein the dissolution-effective amount of mannose ranges from about 1% to about 100% by weight of the tablet.

3. The tablet of claim 1, further comprising a pharmaceutically effective amount of at least one drug, wherein the amount of mannose ranges from about 1% to about 99%.

4. The tablet of claim 1, wherein the tablet hardness exceeds 30 newtons.

5. The tablet of claim 1, wherein the tablet disintegrates within 20 seconds in the presence of less than about 3 mL water.

6. The tablet of claim 1, further comprising a water-soluble diluent selected from the group consisting of sugars, sugar alcohols, polymers, organic acids, organic salts, and inorganic salts.

7. The tablet of claim 6, wherein the diluent is selected from dextrose, fructose, lactitol, lactose, maltitol, maltose, mannitol, sorbitol, sucrose, erythritol, trehalose, dextrates, xylitol, dextrin, maltodextrin, polydextrose, povidone, polyethylene glycol, polyethylene oxide, polyvinyl alcohol, hydroxyethyl cellulose, hydroxyethylmethyl cellulose, hydroxypropyl cellulose, gelatin, citric acid, potassium bicarbonate, potassium chloride, potassium citrate, sodium bicarbonate, sodium chloride, sodium citrate, sodium phosphate dibasic, and sodium phosphate monobasic.

8. The tablet of claim 1, further comprising a highly dispersible diluent selected from the group consisting of agar, kaolin, starch, starch pregelatinized, microcrystalline cellulose, silicified microcrystalline cellulose, powdered cellulose, cyclodextrins, ethylcellulose, chitosan, cellulose acetate, calcium sulfate, calcium carbonate, dibasic calcium phosphate, tribasic calcium phosphate, and carboxymethylcellulose-calcium salt.

9. The tablet of claim 1, further comprising a combination of a carbohydrate and a polymer selected from the group of STARLAC, MICROCELAC, CELLACTOSE, MANNOGEM EZ spray, and combinations thereof.

10. The tablet of claim 1, further comprising at least one disintegrant, sweetener, flavor agent, coloring agent, and lubricant.

11. The tablet of claim 10, wherein the disintegrant is selected from the group consisting of starches, crosslinked polyvinylpyrrolidone, croscarmellose sodium, sodium starch glycolate, and superporous hydrogels.

12. The tablet of claim 10, wherein the sweetener is natural or artificial.

13. The tablet of claim 12, wherein the sweetener is sodium saccharin, aspartame, or cyclamate.

14. The tablet of claim 10, wherein the lubricant is selected from the group consisting of magnesium stearate, stearic acid, polyethylene glycol, talc, sodium stearyl fumarate, colloidal silicon dioxide, and glyceryl behenate.

15. The tablet of claim 1, wherein the mannose is in powder or granulated form.

16. The tablet of claim 15, wherein the granulated form is obtained by wet granulation using a low shear granulator, high shear granulator or fluid bed granulator, and said granulation is conducted at a relative humidity above the critical relative humidity of mannose.

17. A method of administering a drug to a patient, comprising administering to the patient the fast-dissolving tablet of claim 1.

18. A method of making a pharmaceutical tablet having fast-dissolving properties, comprising:
a. providing mannose, and optionally a drug compound in admixture therewith;
b. compressing the mannose-containing composition;
c. subjecting the compressed composition to relative humidity conditions of at least about 50% RH; and
d. drying the humidified, compressed composition to form the tablet.

19. The method of claim 18, wherein said drug compound is admixed with the mannose.

20. The method of claim 19, wherein a non-mannose carbohydrate having low critical relative humidity is combined with the mannose and the drug compound.

21. The method of claim 20, wherein the non-mannose carbohydrate is selected from fructose, dextrates, dextrose, sorbitol and xylitol.

22. The method of claim 18, wherein said compressing is with a force in the range of about 1 MPa to about 50 MPa.

23. The method of claim 18, wherein humidification occurs in a chamber, oven, or room.

24. The method of claim 18, wherein humidification occurs in the presence of constant or variable relative humidity conditions.

25. The method of claim 18, wherein humidification takes place for 1 minute to 24 hours.

26. The method of claim 18, wherein humidification takes place at a temperature in the range of about room temperature to about 60° C.

27. The method of claim 18, wherein said drying is conducted by air drying, vacuum drying, oven drying, or microwave drying.

28. The method of claim 18, wherein the temperature ranges from about room temperature to about 100° C. during the drying.

29. The method of claim 18, wherein humidification and drying are repeated at least once.

30. A fast-dissolving tablet formed by the method of claim 18.
